(19) United States
(12) Patent Application Publication
(10) Pub. No.: US 2012/0296402 A1
(43) Pub. Date: Nov. 22, 2012

(54) DEVICE AND METHOD FOR BROWN ADIPOSE TISSUE ACTIVATION

(76) Inventor: Rebecca Kotter, Shelley, ID (US)

(21) Appl. No.: 13/109,498

(22) Filed: May 17, 2011

(51) Int. Cl. A61F 7/10

(52) U.S. Cl. 607/108

(57) ABSTRACT

The present disclosure is drawn to devices and methods of activating brown adipose tissue. One method comprises applying a cooling device on a subject at a supraclavicular region or paravertebral region of skin overlying brown adipose tissue; and maintaining the cooling device in contact with the skin at a temperature from 45° F. to 70° F. for a duration of at least 90 minutes so as to cool the region sufficiently to activate the brown adipose tissue.

### BACKGROUND

Brown adipose tissue (BAT) was previously thought to exist only in infants, providing non-shivering thermogenesis for protecting an infant's core temperature. In fact, the conventional wisdom was that brown adipose tissue was consumed in infancy after only a few months. However, more recently, with the improvements in medical imaging, “bright spots,” such as those showing up on PET scans, were noticed in many adults, “interfering” with imaging as medical professionals were trying to read these types of scans for other purposes. With the more recent understanding that brown adipose tissue remains active in many adults, research into is its role with respect to adults is also becoming more understood.

### SUMMARY

It has been recognized that enhanced activation of brown adipose tissue in adults can provide many health benefits. In accordance with this, a method of activating brown adipose tissue can comprise applying a cooling device to a subject at a supraclavicular or paravertebral region of skin overlying brown adipose tissue. The method also includes maintaining the cooling device in contact with the skin at a temperature from 45° F. to 70° F. for at least 90 minutes so as to cool the region sufficiently to activate the brown adipose tissue.

In another embodiment, a wearable device for activating brown adipose tissue can comprise a cooling member with an outer insulative layer and an inner conductive layer which is shaped to cover a localized region of skin overlying brown adipose tissue at a supraclavicular region or paravertebral region of a subject.

### BRIEF DESCRIPTION OF THE DRAWINGS

FIG. 1 is a perspective back view of a wearable cooling device prepared in accordance with embodiments of the present disclosure;

FIG. 2 is a front view of the wearable cooling device of FIG. 1;

FIG. 3 is a back view of the wearable cooling device of FIG. 1;

FIG. 4 is a cross-sectional cutaway view, taken along cross-sectional line A-A of FIG. 3, of the wearable cooling device of FIG. 1; and

FIG. 5 is a flow chart depicting a method in accordance with embodiments of the present disclosure.

Additional features and advantages of the disclosure will be apparent from the detailed description which follows, taken in conjunction with the accompanying drawings, which together illustrate, by way of example, features of the disclosure.

### DETAILED DESCRIPTION

Before the present invention is disclosed and described, it is to be understood that this disclosure is not limited to the particular process steps and materials disclosed herein because such process steps and materials may vary somewhat. It is also to be understood that the terminology used herein is used for the purpose of describing particular embodiments only. The terms are not intended to be limiting because the scope of the present invention is intended to be limited only by the appended claims and equivalents thereof.

In describing embodiments of the present invention, the following terminology will be used.

The singular forms “a,” “an,” and “the” include plural referents unless the context clearly dictates otherwise. Thus, reference to a cooling device includes reference to multiple cooling devices, applied cyclically in rotation or applied simultaneously, for example.

The term “subject” refers to a human or other mammal having brown adipose tissue that is treated with a cooling device in accordance with embodiments of the present disclosure.

“Brown adipose tissue” or “BAT” refers to one of the two types of fat tissue found in the body of mammals. Though brown adipose tissue is especially abundant in newborns to generate body heat, it is also present at relatively high concentrations in certain regions of the body in many adults. Brown adipose tissue contains more mitochondria and greater blood supply via capillaries than white adipose tissue. Additionally, brown adipose tissue utilizes both lipids (fats) and glucose (sugar) to generate heat. Thus, there is greater metabolic activity in brown adipose tissue than in some other similar types of tissue.

The term “supraclavicular” refers to the region of the neck mostly on the front of the body that is above the clavicle or collar bone.

The term “paravertebral” refers to the region of the back and neck next to and following the spine. “Cervical paravertebral” refers more specifically to the region that follows the spine next to its cervical portion. “Thoracic paravertebral” refers more specifically to the region following the spine which is next to the thoracic portion thereof.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of 50-250 should be interpreted to include not only the explicitly recited values of 50 and 250, but also include individual values and sub-ranges within the indicated range. Thus, included in this numerical range are individual values such as 60, 70, and 80 micrometers, and sub-ranges such as from 50-100, from 100-200, and from 100-250, etc. This same principle applies to ranges reciting only one numerical value and should apply regardless of the breadth of the range or the characteristics being described.

As used herein, the term “about” means that dimensions, sizes, formulations, parameters, shapes and other quantities and characteristics are not and need not be exact, but may be approximated and/or larger or smaller, as desired, reflecting tolerances, conversion factors, rounding off, measurement error and the like and other factors known to those of skill. Further, unless otherwise stated, the term “about” shall expressly include “exactly,” consistent with the discussion above regarding ranges and numerical data.

It is recognized and described herein that by enhancing or activating the metabolic activity of brown adipose tissue in adults, many health benefits can be achieved. It is further been recognized and described herein that by focusing on regions of the body that include larger concentrations of brown adipose tissue, effective devices and method of increasing health parameters can be achieved efficiently. This can be achieved because brown adipose tissue utilizes both lipids (fats) and glucose (sugar) to generate heat. Thus, by activating this tissue, desired metabolic activity can be achieved when coupled with exercise, or can be achieved passively without exercise. The devices and methods are even more beneficial for subjects that have higher concentrations of brown adipose tissue. For example, though almost all adults have some brown adipose tissue, younger and/or leaner adults tend to have more brown adipose tissue than older and/or more obese adults. That being said, the methods of the present disclosure are effective for both young and/or lean subjects, as well as older or more obese subjects.

PET scans or other imaging techniques can be used to specifically identify where the greatest concentrations of brown adipose tissue can be found, but more practically, for most adults, the heaviest concentration of brown adipose tissue can be found in the supraclavicular region as well as the paravertebral region. Thus, in accordance with embodiments of the present disclosure, a method of activating brown adipose tissue can include applying cooling temperatures to the supraclavicular and/or paravertebral region(s) to activate brown adipose tissue.

More specifically, a method of activating brown adipose tissue can comprise applying a cooling device to a subject at a supraclavicular region or paravertebral region of skin overlying brown adipose tissue. The method further includes maintaining the cooling device in contact with the skin at a temperature from 45° F. to 70° F. for a duration of at least 90 minutes so as to cool the region sufficiently to activate the brown adipose tissue.

The cooling device can be any cooling device configured for application to the supraclavicular region or the paravertebral region, such as an ice pack, an electric cooling device, a refrigerant cooling device, or the like. In one example, a particular cooling device is specifically disclosed in accordance with one particular embodiment. Specifically, a wearable device for activating brown adipose tissue can comprise a cooling member with an outer insulative layer and an inner conductive layer which is shaped to cover a localized region of skin overlying brown adipose tissue at a supraclavicular region or paravertebral region of a subject.

With these specific embodiments in mind, it is notable that in discussing the methods and devices described herein, discussion each example is also applicable to the other example. Thus, when explicitly describing method embodiments, that discussion is translatable in context to device embodiments, and vice versa.

In accordance with examples of the present disclosure, the duration of daily application can be at least 90 minutes, as previously described; however, often the application time can be from 2 hours to 12 hours. It takes an amount of time to fully or substantially activate brown adipose tissue, and the benefits of the present disclosure can be more readily achieved after the brown adipose tissue is activated more fully and the cooling is maintained once the tissue has reached this state. In another embodiment, the method can comprise repeatedly applying the cooling device as prescribed herein on from 4 to 7 days of a week over a period of at least a month. In still another embodiment, multiple applications in a single 24 hour (daily) period can also provide benefits. In this embodiment, the method can comprise repeatedly applying the cooling device for a duration of 2 hours to 6 hours per cycle at from 2 to 4 times per day. This has practical advantages if it takes time to re-cool a given cooling device for use during a second or third cycle, etc. In one example, multiple devices can be used and alternated for application, e.g., one is used while the other is being prepared in a freezer apparatus for use. Alternatively, if using an electric or refrigerant cooling device that is adapted for supraclavicular or paravertebral application, this consideration may not be as relevant, depending on the technology. It is also noted that there may be some benefit to allowing the skin to warm to room temperature between cycles, to give the tissue time to rest before reactivation. Regardless of the cooling device used, or the various timing options selected for treatment, in one specific embodiment, the cooling device can be adapted to cool the specific region of application by at least 20° F. within 15 minutes, though this is not required.

As mentioned, the application region can be the supraclavicular region or the paravertebral region. However, in one embodiment, the cooing device can be configured for application over both regions simultaneously. In another embodiment, the paravertebral region can include a cervical paravertebral region and/or a thoracic paravertebral region. If a specific subject knows more precisely where the brown adipose tissue is concentrated within the subject's own anatomy, the cooling device can be adapted more specifically to that subject. However, for most individuals, that tissue will be concentrated in the areas described herein.

There are many health benefits of activating brown adipose tissue in accordance examples of the present disclosure. One practical benefit is that of systemic weight loss. Specifically, by activating brown adipose tissue, serum levels of glucose and lipid levels can be dropped in a subject. Other benefits of the treatments described herein can include reduced adiposity, enhanced metabolic rate, reduced serum glucose, reduced insulin resistance, and/or improved lipid profile. Other secondary benefits can include improved (usually lowered) blood pressure, improved cardiovascular risk profile, and/or reduction in the need for anti-diabetic medication and anti-lipid medication.

Turning now to specific cooling devices that are effective in accordance with embodiments of the present disclosure, it is emphasized that this specific device shown in the FIGS. is not necessary for use in carrying out the methods of the present disclosure. This device (as will be discussed more fully below) is provided and adapted for the methods of the present disclosure because it can retain its coolness for a prolonged period of time, can be more flexible at the surface for comfort, and can maintain an appropriate level of cooling for activating brown adipose tissue. Electric cooling devices, refrigerant-based cooling devices, other types of cooling pads or ice packs, or the like can also be used. For example, technology exists in small packages for cooling laptops and other electronic devices, as well as for cooling animals and humans, e.g., cooling for mattresses and cooling blankets or pillows. To illustrate the flexibility of the methods described herein, an electric or refrigerant system in a cooling pillow could be used to cool the neck and upper back region while a subject sleeps, or could be used on a high back chair or recliner while a subject cools the back of the neck and upper spine while watching television or reading. This would be an embodiment where the paravertebral region is primarily cooled.

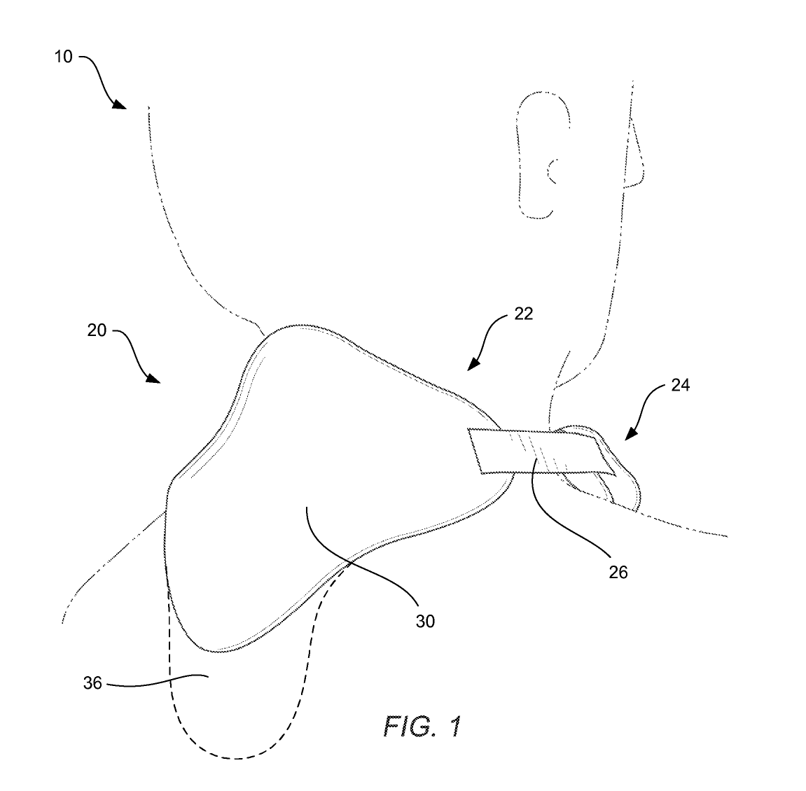

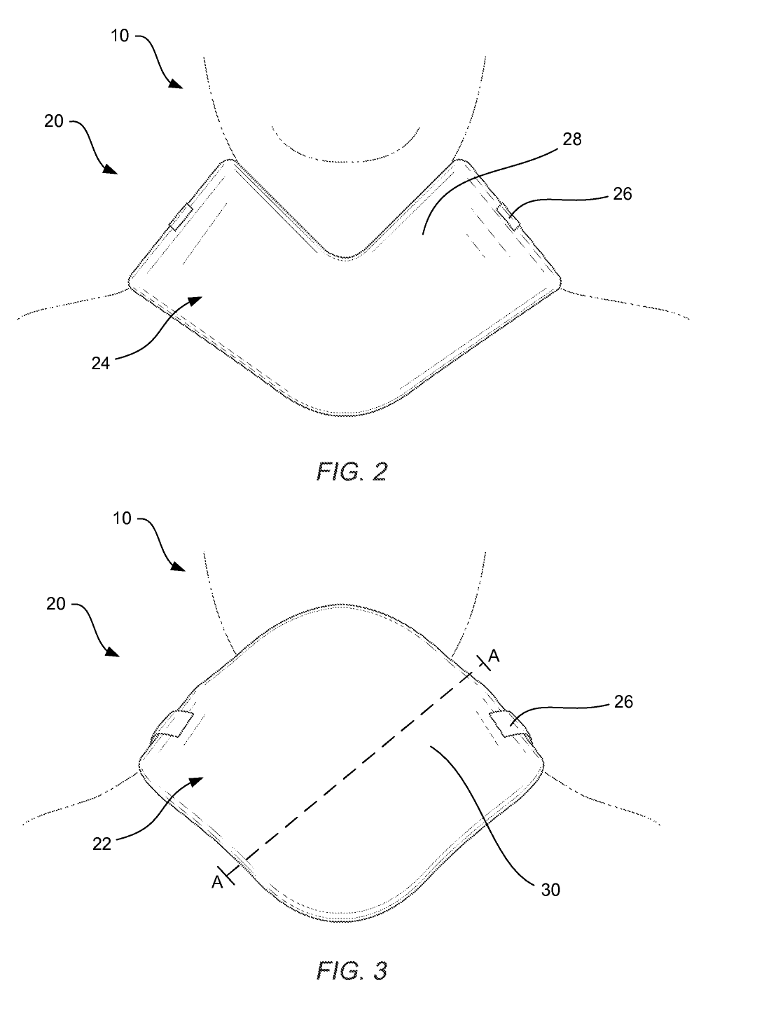

In FIGS. 1-3, a cooling device 20 is shown that is portable and can be worn relatively comfortably on a subject 10 during normal activities. Specifically, the device includes a back section 22 and a front section 24. In this embodiment, the front section shaped to cover a supraclavicular front region of the subject and the back section shaped to cover a paravertebral back region of the subject, though this configuration is not intended to be limiting. As shown, with respect to the paravertebral back region of the subject, the coverage provided in this embodiment is primarily to the cervical paravertebral back region, though it could be extended further down to the thoracic paravertebral back region, as shown at 36.

Also shown in FIGS. 1-3, the back section 22 and the front section 24 are connected together by a flexible or even adjustable strap 26. The strap provides a mechanism of connecting the back section to the front section, and thus hold the device in place around the neck region, as shown. However, the device could be fastened and held in place by any of a number of methods, such as with snaps, velcro, buttons, clasps, clips, or any other workable fastener. Likewise, the device could alternatively be configured to be wrapped more entirely around the neck region, being connected by a fastener at only one location (rather than on both sides, as shown). Alternatively, the cooling device could be memory shaped or spring loaded so that it remains in place without the need of a fastener.

As shown, the device provides coverage to skin over concentrated areas of brown adipose tissue in the supraclavicular region 28, and a paravertebral region 30. Other areas can also be covered to activate additional brown adipose tissue, but these regions more universally represent locations within many subjects where brown adipose tissue is found at higher concentrations than in other places in the body. In the embodiment shown in FIGS. 1-3, the region where the cooling device is applied has an area of about 200 cm² to about 500 cm², though different sizes or configurations are also usable.

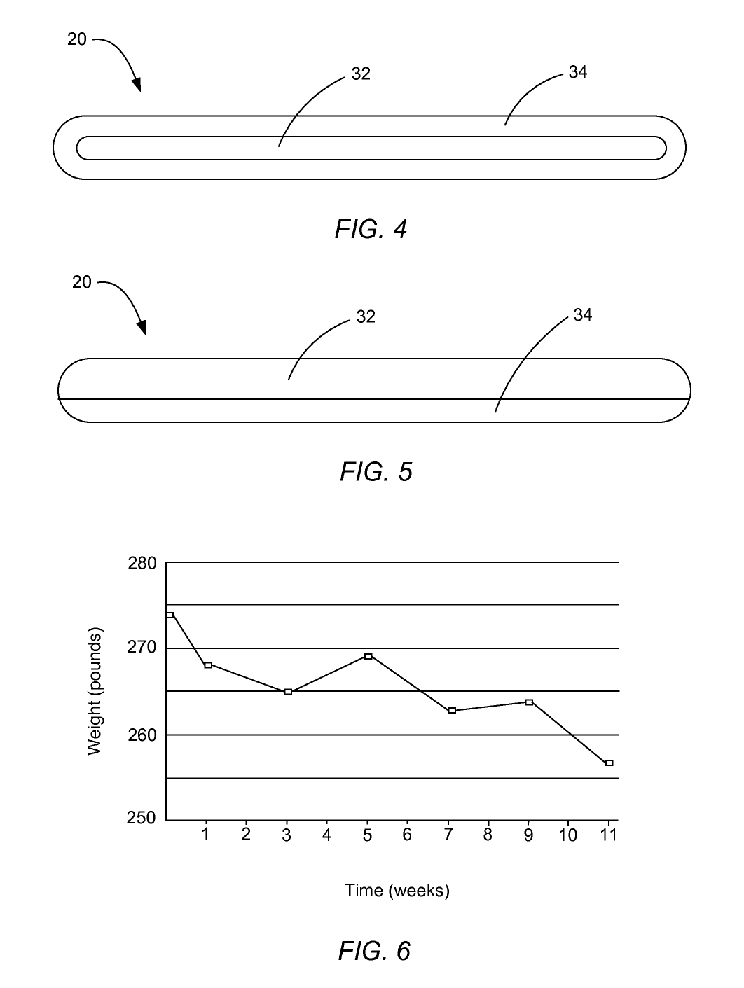

Turning now specifically to FIGS. 4 and 5, these FIGS. provide alternative cross-sectional embodiment views of the cooling device of FIG. 3, taken along cutaway section A-A. In FIG. 4, the cooling device 20 is revealed to be a core-shell, dual-chambered system. More specifically, the cooling device includes an inner conductive layer 32 an outer insulative layer 34. FIG. 5 is similar, except that the cooling device 20 as shown is a bi-layer, dual chambered system. Specifically, the cooling device includes a distal conductive layer 32 (with respect to skin contact) and a proximal insulative layer 34 (with respect to skin contact). In these embodiments, the device comprises a thin, fluid-filled insulative layer that lies against the skin. This insulative layer can contain a concentrated saline solution and will remain as a fluid even when the conductive layer is frozen. Some purposes for this insulative layer include achieving maximum skin conformity right out of the freezer, and providing comfort to the subject while in use. The conductive layer (inner conductive layer of FIG. 4 or the distal conductive layer of FIG. 5) can be thicker that the insulative layer (at least a portion of which is more proximal to the skin than the insulative layer), and typically form a solid ice layer which melts slowly and provides cooling long after the insulative layer layer has lost its own cool temperature. Thus, the long term cooling provided by the conductive layer can be transferred to the insulative layer, and ultimately to the skin, providing long lasting and comfortable cooling of the skin.

In examples according to FIGS. 4 and 5, as mentioned, the conductive layer can contain water and the insulative layer can contain a hypertonic salt solution, e.g., 20 wt % to 22 wt % sodium chloride and water, though other solutions or compositions can be used. As an alternative, gel-like material might be used in the outer insulative layer to make the device more comfortable to wear, or to regulate the cooling within a desired predetermined range. In one embodiment, the dual chamber system can be configured and formulated to cool the supraclavicular and/or paravertebral region by 20-25° F. below a room temperature baseline in the first 15 minutes of use (contrasted to the single-chamber ice pack which may only cool the supraclavicular region by about 10° F. in the first 15 minutes of use). Furthermore, a dual chamber system can be formulated to maintain supraclavicular and/or paravertebral regional temperature in the 50° F. to 70° F. range for at least 90 minutes, 2 hours, 3 hours, 4 hours, or more, without the requirement of changing out the cooling device.

One of the advantages of the methods of present disclosure is that of efficacy, even in the absence of diet modification and/or exercise. However, moderate diet and/or moderate exercise can assist in weight loss or other health benefits as described herein. More extreme diet and more extreme exercise regimens can have a negative impact on brown adipose tissue, and thus, moderate exercise and/or moderate diet may be more effective in combination with the methods of the present disclosure than more heavy diet and/or extreme exercise regimens. Furthermore, eating also will tend to activate brown adipose tissue, so moderate eating in combination with the implementation of the methods of the present disclosure can also assist in weight loss or other health benefits. One possible regimen may be to eat smaller meals more frequently than 3 times a day, thus keeping the brown adipose tissue activated as long as possible while providing cooling in accordance with examples of the present disclosure.

### EXAMPLES

### Example 1

A female adult weighing approximately 272 pounds for a period of about 10 years wore a cooling device over the supraclavicular and cervical paravertebral regions for a period of several months. During this period of time, no changes were made with respect to typical diet or exercise. The only difference was the almost daily use (averaging 6 hours per day, 5 days per week) of the cooling device. FIG. 5 provides weight loss data showing the effectiveness of this treatment over an 11 week period.

### Example 2

A female adult weighing approximately 253 pounds wore a cooling device in accordance with embodiments of the present disclosure for a period of about seven (7) months, with application of about 30 hours per week (4-7 times per week). During this period of time, no significant changes were made with respect to typical diet or exercise, and if anything, because of noticeable weight loss, the subject increased caloric intake occasionally. After a period of seven months, the subject's weigh was reduced to 240 pounds.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the claims set forth below.

1. A method of activating brown adipose tissue, comprising:
applying a cooling device on a subject at a supraclavicular region or paravertebral region of skin overlying brown adipose tissue; and
maintaining the cooling device in contact with the skin at a temperature from 45° F. to 70° F. for a duration of at least 90 minutes so as to cool the region sufficiently to activate the brown adipose tissue.

1. The method of claim 1, wherein the duration is from 2 hours to 12 hours.

3. The method of claim 2, further comprising repeatedly applying and maintaining the cooling device in contact with the skin for 4 to 7 days per week over a period of at least a month.

4. The method of claim 1, further comprising repeatedly applying and maintaining the cooling device in contact with the skin cyclically for 2 hours to 6 hours at 2 to 4 times per day.

5. The method of claim 4, wherein the skin is allowed to warm to room temperature between each cycle.

6. The method of claim 1, wherein the cooling device cools the skin by at least 20° F. within 15 minutes.

7. The method of claim 1, wherein the cooling device is applied to both the supraclavicular region and the paravertebral region.

8. The method of claim 1, wherein the paravertebral region includes a cervical paravertebral region.

9. The method of claim 1, wherein the paravertebral region includes a thoracic paravertebral region.

10. The method of claim 1, wherein the cooling device has a skin cooling area of about 200 cm² to about 500 cm².

11. The method of claim 1, wherein activating brown adipose tissue is continued until a predetermined weight loss is achieved.

12. The method of claim 1, wherein activating brown adipose tissue in the subject results in at least one of reduced adiposity, enhanced metabolic rate, reduced serum glucose, reduced insulin resistance, or improved lipid profile.

13. The method of claim 1, wherein activating brown adipose tissue in the subject results in at least one of lowered blood pressure, improved cardiovascular risk profile, or reduction in the need for anti-diabetic medication and anti-lipid medication.

14. A wearable device for activating brown adipose tissue, comprising a cooling member comprising an insulative layer and a conductive layer and shaped to cover a localized region of skin overlying brown adipose tissue at a supraclavicular region or paravertebral region of a subject, wherein at least a portion of the insulative layer is configured to be more proximally located with respect to the skin than the conductive layer.

15. The device of claim 14, wherein the cooling member comprises a front section and a back section, the front section shaped to cover the supraclavicular region and the back section shaped to cover the paravertebral back region.

16. The device of claim 15, wherein the front section and the back section are held together with flexible or adjustable strap.

17. The device of claim 14, wherein the conductive layer includes water and the insulative layer includes a hypertonic salt solution.

18. The device of claim 17, wherein the hypertonic salt solution comprises 20 wt % to 22 wt % sodium chloride and water.

19. The device of claim 14, wherein the insulative layer and the conductive layer have a bi-layer configuration.

20. The device of claim 14, wherein the insulative layer and the conductive layer have a core-shell configuration.
